(19) United States
(12) Patent Application Publication
(10) Pub. No.: US 2019/0092902 A1
(43) Pub. Date: Mar. 28, 2019

(54) POLYPEPTIDE COPOLYMER, POROUS FIBROUS SCAFFOLD INCLUDING THE SAME AND METHOD FOR NERVE REGENERATION OR GROWTH

(71) Applicant: NATIONAL TAIWAN UNIVERSITY, Taipei City (TW)

(72) Inventors: Wei-Fang SU, Taipei City (TW); Ta-Ching CHEN, Taipei City (TW)

(21) Appl. No.: 16/142,051

(22) Filed: Sep. 26, 2018

Related U.S. Application Data
(60) Provisional application No. 62/563,106, filed on Sep. 26, 2017.

(51) Int. Cl. C08G 69/10; C12N 5/00; C12N 5/079; A61L 27/54; A61L 27/58; A61L 27/56; A61L 27/22; D01F 4/00; D01D 5/00

(52) U.S. Cl. CPC C08G 69/10; D10B 2211/20; C12N 5/0621; A61L 27/54; A61L 27/58; A61L 27/56; A61L 27/227; D01F 4/00; D01D 5/0038; C12N 2501/13; C12N 2533/30; C12N 2537/00; A61L 2430/32; D10B 2509/00; D10B 2401/12; C12N 5/0068

(57) ABSTRACT

A polypeptide copolymer, a preparation method thereof, a porous fibrous scaffold including the same, and a method for nerve regeneration or growth are disclosed. The polypeptide copolymer comprises: a glutamate unit; and a glutamic acid unit, wherein a ratio of a content of the glutamic acid unit to a content of the glutamate unit is in a range from 10:90 to 90:10.

### CROSS REFERENCE TO RELATED APPLICATION

This application claims the benefit of filing date of U.S. Provisional Application Ser. No. 62/563,106, entitled “Synthesis and fabrication of neuron cue containing polypeptide for neuron regeneration 3D scaffolds” filed Sep. 26, 2017 under 35 USC § 119(e)(1).

### BACKGROUND OF THE INVENTION

### 1. Field of the Invention

The present invention relates to a polypeptide copolymer, a preparation method thereof, a porous fibrous scaffold including the same, and a method for nerve regeneration or growth, and more particularly, to a random copolymer comprising a glutamate unit and a glutamic acid unit, a preparation method thereof, a porous fibrous including the same, and a method for nerve regeneration or growth.

### 2. Description of Related Art

The average life expectancy of human beings has been greatly increased with medical advances. However, the neural tissue damage is still one of the leading causes of disability and death. Neural tissue dysfunction includes stroke, traumatic injury of brain, eye, and spinal cord, and neurodegenerative diseases such as Alzheimer's disease, Parkinson's disease, and Huntington's disease.

Traditionally, neuron cells plus supplementation of growth factor are used to stimulate neurite regeneration, but the effect does not meet expectations. Recently, scientists have dedicated themselves to the study of neural tissue engineering, which is considered to have the potential to restore the damaged neural tissue. The tissue engineering is mainly to design a scaffold for the mechanical properties and high biocompatibility, and to create a suitable growth environment for cells. The purpose is to transplant the scaffold and tissues into a subject after the in vitro cells develop into the tissues with functions.

Polymers commonly used in the tissue engineering can be classified into natural polymers and synthetic polymers. Natural polymers have advantages such as excellent biocompatibility and cell adhesion, but its disadvantages often include poor mechanical properties, difficulty in controlling the degradation rate, and greater differences between batches. However, the synthetic polymers have excellent mechanical properties, adjustable degradation rate, and hydrophobic or hydrophilic properties. But, a surface modification is needed for the synthetic polymers to increase the cell adhesion and cell growth, and the synthetic polymers cause immune responses easily and damage the existing tissues after degradation.

Therefore, it is desirable to provide a new polymer that not only has excellent biocompatibility but also excellent mechanical properties, cell adhesion, ability to decrease immune responses, or ability to stimulate neurite growth, and the new polymer can be applied to the field of nerve regeneration or growth.

### SUMMARY OF THE INVENTION

In the light of this, the present invention provides a novel material of a polypeptide copolymer having excellent biocompatibility, adjustable hydrophilicity and hydrophobicity, or ability to stimulate neurite growth so as to be applied in nerve regeneration or growth.

To achieve the object, the present invention provides a polypeptide copolymer, comprising a glutamate unit; and a glutamic acid unit; wherein a ratio of a content of the glutamic acid unit to a content of the glutamate unit is in a range from 10:90 to 90:10.

The present invention may adjust the hydrophilicity and hydrophobicity by introducing a neurotransmitter glutamate into the polymer backbone. However, if the concentration of glutamic acid is too high, the polypeptide copolymer will be insoluble in a common organic solvent, and thus affects its processability. Besides, the high concentration of glutamic acid may cause cytotoxicity leading to apoptosis. Preferably, the ratio of the content of the glutamic acid unit to the content of the glutamate unit is in a range from 10:90 to 40:60 or from 10:90 to 30:70, and more preferably from 15:85 to 40:60 or from 15:85 to 30:70 according to an embodiment of the present invention. However, the present invention is not limited thereto. For example, the ratio of the content of the glutamic acid unit to the content of the glutamate unit may be in a range from 15:85 to 35:65, from 10:90 to 50:50, from 15:85 to 60:40, or from 10:90 to 35:65.

In the present invention, the polypeptide copolymer is a random copolymer. The weight average molecular weight of the polypeptide copolymer can be in a range from 100 kDa to 500 kDa, preferably from 150 kDa to 300 kDa, and more preferably from 200 kDa to 300 kDa. However, the present invention is not limited thereto. For example, the weight average molecular weight of the polypeptide copolymer may be in a range from 150 kDa to 280 kDa, from 170 kDa to 250 kDa, from 180 kDa to 250 kDa, from 180 kDa to 280 kDa, or from 180 kDa to 300 kDa.

The polypeptide copolymer of the present invention has a structure of following formula (I):

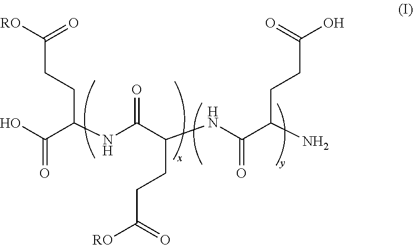

wherein R can be C₁₋₆ alkyl group or benzyl group, and x:y=10:90 to 90:10.

In the present invention, the glutamate (acid) unit can be a benzyl glutamate (acid) unit, a methyl glutamate (acid) unit, an ethyl (acid) glutamate unit, a propyl (acid) glutamate unit, a butyl (acid) glutamate unit, a pentyl (acid) glutamate unit or a hexyl (acid) glutamate unit. Preferably, the glutamate (acid) unit is a benzyl glutamate (acid) unit.

In the polypeptide copolymer described in the present invention, the amount of glutamic acid affects the properties of the entire polypeptide copolymer. Therefore, how to control the content of glutamic acid in the polypeptide copolymer is important. If the preparation is carried out by condensation using glutamic acid monomers and glutamate monomers in different ratios, the molecular weight of the product will not be easily controlled. Moreover, since the glutamic acid has two different types of acid group, it leads to a complicated structure of the product and poor reproducibility. As a result, the present invention provides a method for preparing the aforementioned polypeptide copolymer, comprising steps of: providing a polyglutamate; and reacting the polyglutamate with an acid for a predetermined period of time to obtain the polypeptide copolymer. In the preparation method provided by the present invention, the polypeptide copolymer with different ratios of the units may be obtained by controlling the predetermined period of time. However, the polypeptide copolymer of the present invention is not limited to be prepared by the method of the present invention.

In an embodiment of the present invention, said polyglutamate is poly(γy-benzyl-L-glutamate). However, the present invention is not limited thereto. For example, the polyglutamate may be poly(γ-methyl-L-glutamate), poly(γ-ethyl-L-glutamate), poly(γ-propyl-L-glutamate), poly(γ-butyl-L-glutamate), poly(γ-pentyl-L-glutamate), or poly(γ-hexyl-L-glutamate). In the present invention, said polyglutamate preferably is poly(benzyl glutamate), such as poly(γ-benzyl-L-glutamate).

In an embodiment of the present invention, the weight average molecular weight of the polyglutamate can be in a range from 100 kDa to 500 kDa, preferably from 160 kDa to 350 kDa, and more preferably from 200 kDa to 300 kDa. However, the present invention is not limited thereto. For example, the weight average molecular weight of the polyglutamate may be in a range from 160 kDa to 300 kDa, from 170 kDa to 320 kDa, from 180 kDa to 320 kDa, from 160 kDa to 320 kDa, or from 170 kDa to 300 kDa.

In the present invention, said “acid” includes an organic acid, an inorganic acid or a combination thereof. For example, the acid may be trifluoroacetic acid, hydrochloric acid, sulfuric acid, or hydrobromic acid. However, the present invention is not limited thereto as long as the glutamate can be hydrolyzed into glutamic acid. In an embodiment of the present invention, the acid is hydrobromic acid.

In the present invention, the “predetermined period of time” is not particularly limited. Theoretically, the longer the predetermined period of time, the higher the content of the glutamic acid hydrolyzed from glutamate. Therefore, the predetermined period of time can be adjusted in order to meet the needs of the product and the amount of the reactant as long as the glutamate with predetermined ratio can be hydrolyzed to glutamic acid within the predetermined period of time. For example, it may be in a range from 5 to 60 minutes, from 10 minutes to 25 minutes, from 20 minutes to 45 minutes, from 10 minutes to 50 minutes, from 1 hour to 5 hours, or from 3 hours to 10 hours. However, the present invention is not limited thereto.

The present invention further provides a porous scaffold, comprising the aforementioned polypeptide copolymer. In an embodiment of the present invention, the porous scaffold may be obtained from the polypeptide copolymer by electrospinning. However, the present invention is not limited thereto. For example, the porous fibrous scaffold may be prepared by phase separation, solvent casting/particulate leaching, or gas foaming.

In the present invention, the porous scaffold obtained from electrospinning process is called porous fibrous scaffold may comprise an isotropic fiber or aligned fiber. In an embodiment of the present invention, the porous fibrous scaffold has a structure of unidirectional arrangement, and it can lead the neurites grow in a specific direction. Hence, it has better clinical application potential.

In the present invention, a fibrous scaffold is a porous fibrous scaffold. Since the porous fibrous scaffold has a 3D space for cell adhesion, the cell can migrate into the porous fibrous scaffold in order to enhance the cell adhesion.

The porous fibrous scaffold of the present invention can be used as a biological scaffold for tissue engineering. It is especially useful for nerve regeneration and/or growth. Therefore, the present invention also provides a method for nerve regeneration and/or growth, comprising a step of: seeding a nerve cell onto a porous fibrous scaffold, wherein the porous fibrous scaffold comprises a glutamate unit and optionally further comprises a glutamic acid; when the porous fibrous scaffold comprises the glutamate unit and the glutamic acid, a ratio of a content of the glutamic acid unit to a content of the glutamate unit is in a range from 10:90 to 90:10. Since the polypeptide copolymer of the present invention is used to form a 3D porous fibrous scaffold by electrospinning, it can be used as a biomimetic and hierarchical structure of extracellular matrix (ECM). Therefore, the nerve cells are in a 3D environment to reconstruct the innate structure-function relationship through cell adhesion and cellular interactions so as to achieve nerve regeneration and/or growth.

In one embodiment of the present disclosure, it was found that the porous fibrous scaffold made from polyglutamate can induce neurite growth, and has a longer neurite than that of a group not using a fibrous scaffold.

In an embodiment of the present invention, it was found that the obtained porous fibrous scaffold has excellent biocompatibility when the side chain hydrolysis rate of the polyglutamate is about 20% and 30%. Besides, compared to the porous fibrous scaffold with no glutamic acid unit introduced, the abovementioned porous fibrous scaffold can induce more neurites or longer neurites, and a tight network of the cells can be formed. Hence, the introduction of glutamic acid into the polypeptide copolymer backbone of the present invention facilitates the induction of neural differentiation.

The nerve cells according to the present invention are central nerve cells, including spinal nerve cells, cranial nerve cells, optic nerve cells, olfactory nerve cells, or facial nerve cells. However, the present invention is not limited thereto.

In addition, since the porous fibrous scaffold of the present invention can induce neural differentiation and/or neurite growth, it may be further applied to treat or ameliorate neurodegenerative diseases (e.g., Alzheimer's disease, Parkinson's disease, Huntington's disease, amyotrophic lateral sclerosis (ALS), spinal muscular atrophy, Spinocerebellar Ataxia, spinal cord injury, or head trauma. However, the present invention is not limited thereto.

### BRIEF DESCRIPTION OF THE DRAWINGS

FIG. 1 is an electrospinning system according to the present invention.

FIG. 2 is SEM images of porous fibrous scaffolds made from isotropic PBG fibers according to an embodiment of the present invention.

FIG. 3 is SEM images of porous fibrous scaffolds made from aligned PBG fibers according to an embodiment of the present invention.

FIG. 4A is a bar chart of a biocompatibility test for porous fibrous scaffolds made from isotropic fibers according to one embodiment of the present invention.

FIG. 4B is a bar chart of a biocompatibility test for porous fibrous scaffolds made from aligned fibers according to an embodiment of the present invention.

FIG. 5A is SEM images of porous fibrous scaffolds made from isotropic fibers with cells adhered thereon according to an embodiment of the present invention.

FIG. 5B is SEM images of porous fibrous scaffolds made from aligned fibers with cells adhered thereon according to an embodiment of the present invention.

FIG. 6 is a bar chart of adsorption of poly-L-lysine by porous fibrous scaffolds according to an embodiment of the present invention.

FIG. 7 is a graph showing the weight loss with time for the porous fibrous scaffolds in the biodegradability test according to an embodiment of the present invention.

FIG. 8 is a graph showing changes in pH of porous fibrous scaffolds during degradation according to an embodiment of the present invention.

FIG. 9A is images of immunofluorescence staining of PC-12 with the porous fibrous scaffolds made from isotropic fibers according to an embodiment of the present invention

FIG. 9B is images of immunofluorescence staining of PC-12 with the porous fibrous scaffolds made from aligned fibers according to an embodiment of the present invention.

FIG. 10A to FIG. 10C are SEM images showing the cells on porous fibrous scaffolds made from PBGA₃₀-A fibers at different differentiation time point according to an embodiment of the present invention.

FIG. 11 is an SEM image of a hiPSC derived RGC on a porous fibrous scaffold made from PBGA fibers.

FIG. 12A to 12C are staining diagrams of a hiPSC-derived RGC optic vesicle (OV) structure according to an embodiment of the present invention

FIG. 12D is a bar chart of neurite lengths of the hiPSC-derived RGC according to an embodiment of the present invention.

### DETAILED DESCRIPTION OF THE PREFERRED EMBODIMENT

The following specific examples are used to illustrate the present invention. Any person who is skilled in the art can easily conceive other advantages and effects of the present invention. Although the present invention has been explained in relation to its preferred embodiment, many other possible modifications and variations can be made without departing from the spirit and scope of the present invention as hereinafter claimed.

Materials and Instrument

L-glutamic acid γ-benzyl ester: 99% purity, commercially available from Aldrich

Triphosgene: 98% purity, commercially available from Aldrich

Sodium: 99% purity; commercially available from Aldrich

Dichloroacetic acid: 99% purity, commercially available from Acros

33 wt % HBr in acetic acid: 99% purity, commercially available from Acros

Rat adrenal medulla pheochromocytoma (PC-12): provided by Wei-Fang, Su lab

Human episomal induced pluripotent stem cell (hiPSC): provided by Wei-Fang, Su lab

Nuclear magnetic resonance spectroscopy: Bruker DPX400

Scanning electron microscope (SEM): JEOL JSM-6700F

Gel permeation chromatography: Waters Breeze 2

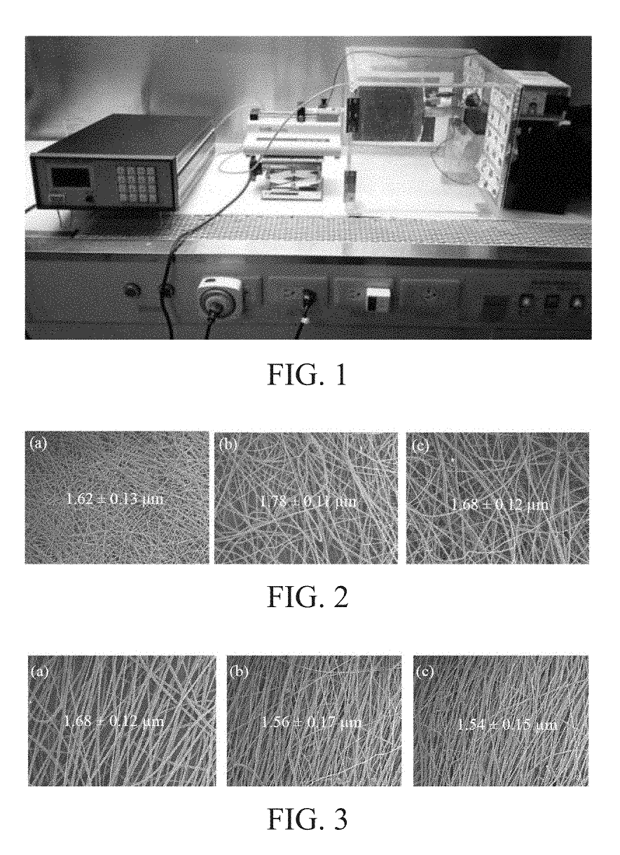

Electro-spinning system: FIG. 1, commercially available from Sunway Scientific Corporation

Contact angle meter: Sindatek Model 100SB

Quartz crystal microbalance: QCM-D E1 Biolon Scientific

Sample Preparation and Characterization

Nuclear magnetic resonance spectroscopy is used for structure characterization of monomers and polymers.

Sample formulation: (1) A monomer (γ-benzyl glutamate-N-carboxy anhydride, 5-6 mg) was added into 800 μL of chloroform-d. (2) A monomer (polypeptide copolymer, 10 mg) was added into 800 μL of trifluoroacetic acid-d (TFA-d).

Contact Angle Measurement

The contact angle was measured and calculated by the contact angle meter and the following formula (I).

γ_{SL}+γ_{LG}×cos θ=γ_{SG} (I)

Adhesion Test of the Porous Fibrous Scaffold

The protein adhesion instrument used in the present invention is a quartz crystal microbalance. The gold piece was pasted on an aluminum foil, electro-spinning was carried out for 3 minutes, and then the gold piece was vacuumed overnight to remove the solvent. The gold piece was detached from the foil, and the adhesive agent on the gold piece was removed by acetone. Afterwards, the gold piece was placed in the instrument, and bubbles among the fibers were taken away by a high flow rate of deionized water. The flow rate was decreased when there is no bubble formed. Keep waiting for 1-3 hours until the water was adsorbed onto the surface of fibers to reach equilibrium. Finally, a low flow rate of poly-L-lysine was used for 1-3 hours. The adhesive amount of the poly-L-lysine was calculated when the frequency stopped changing.

Biodegradation Test of the Porous Fibrous Scaffold

The vacuumed and dried polymeric fiber film was cut into an appropriate size, and an initial weight of the film was W₀. Then the foil was placed in a 24-well plate, added with 1 mL of phosphate buffer solution, and then saved in a cell incubator at 37° C. The plate was taken out of the cell incubator at a specific time point. The film was washed back and forth with distilled water to ensure the salt was completely dissolved, placed in a vacuum oven at 40° C. for 12 hours, and then taken out to measure the weight W, representing the weight after degradation. The degradation percentage was calculated by the following formula (II).

Remaining weight (%)=100×W/W₀ (II)

Determination of pH After Porous Fibrous Scaffold Degradation

The vacuumed and dried polymeric fiber film was cut into an appropriate size and placed in a 24-well plate, added with 1 mL of a phosphate buffer solution, and then saved in a cell incubator at 37° C. The plate was taken out of the cell incubator at a specific time point to measure the pH of the solution.

Cell Culture and Cell Differentiation

The PC-12 culture medium was formulated according to the following Table 1. A culture plate was coated with 2 mL of poly-L-lysine to facilitate cell attachment and growth. After 10 minutes, the poly-L-lysine which was not attached to the plate was removed, and then the plate was used for cell culture. Thereafter, 15,000 PC-12 cells were added into each well of the 24-well plate, and cultured for 2 days. After confirming the cells were attached to the substrate, the culture medium was replaced with differentiation medium, and 100 ng/mL nerve growth factor (NGF) was added, wherein the components of the differentiation medium are shown in the following Table 1.

TABLE 1

| Component | Concentration | |
|---|---|---|
| DMEM high glucose | 1 | pack/L |
| Sodium bicarbonate | 1.5 | g/L |
| Horse serum | 10% | |
| Fetal bovine serum | 5% | |
| Streptomycin | 0.1 | g/L |
| Penicillin | 0.06 | g/L |

Biocompatibility Test of the Porous Fibrous Scaffold

The porous fibrous scaffold was placed in a 24-well plate, and the plate was immersed in 70% alcohol for 3 hours, followed by sterilized by ultraviolet light for 12 hours. Next, 5,000 cells (PC-12) were seeded onto each porous fibrous scaffold, 1 mL of the culture medium was added, and the plate was cultured in an incubator. The present invention is mainly carried out by the MTT test, and the MTT solution concentration was 5 mg/mL PBS. At a specific time point, 100 μL of MTT solution was added into the 24-well plate and the plate was placed in an incubator for 2 hours. After the reaction, the MTT solution was removed, and then 200 μL of DMSO solution was added, and the plate was evenly shaken to completely dissolve the crystallization. Lastly, 100 μL of the purple solution was added to a 96-well plate, and an absorbance (λ=570 nm) was measured by an ELISA reader.

Synthesis of Polypeptide Copolymer

Synthesize a polypeptide copolymer according to the synthetic route of Scheme I below.

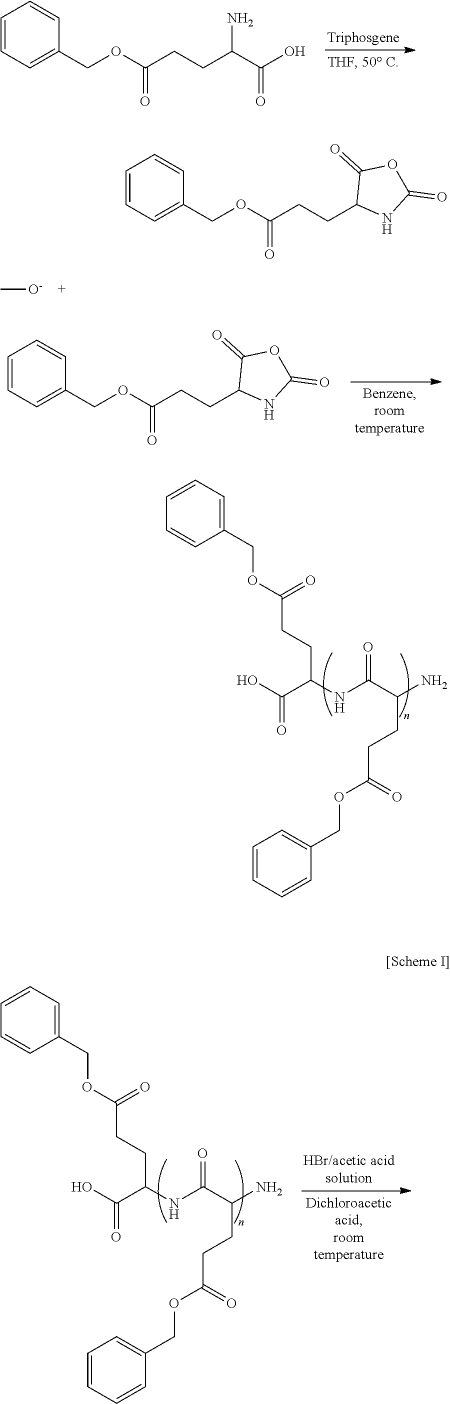

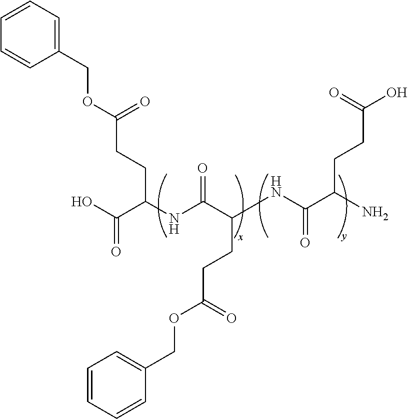

Step 1: Synthesis of γ-benzyl glutamate-N-carboxy anhydride (benzyl-Glu-NCA)

L-glutamic acid γ-benzyl ester (5 g) and triphosgene (3.13 g) were respectively added to a 500 mL and 250 mL round-bottomed flask with a molar ratio of 2:1. Then, 200 mL and 20 mL of anhydrous tetrahydrofuran (THF) were respectively added. After the triphosgene was completely dissolved, the solution containing triphosgene was transferred to the 500 mL round-bottomed flask by using a double-ended needle. The mixture reacted at 50° C. for 1 hour until the solution was clear. Thereafter, hexane (2000 mL) was added and the mixture recrystallized for 3-5 times at −20° C. to purify the product. Lastly, the product was placed in a vacuum oven overnight to remove the solvent for the next step.

¹H NMR (400 MHz, CDCl3) δ 2.14 (m, 2H), 2.61 (m, 2H), 4.39 (t, 1H), 5.15 (s, 2H), 6.4 (s, 1H), 7.37 ppm (s, 5H).

Step 2: Synthesis of poly(benzyl glutamate) (PBG)

The reaction vessel was evacuated for 12 hours, and 4 Å molecular sieves were added to the reaction solvent benzene and then filled with nitrogen gas overnight to remove water. Next day, benzyl-Glu-NCA (5 g) was provided in a 500 mL round-bottomed flask, and the round-bottomed flask was evacuated afterwards. In addition, a small piece of sodium (40 mg) was provided in a 250 mL round-bottomed flask, added with anhydrous methanol (5 mL) by using gas-tight syringe, and nitrogen gas kept flowing into the flask during the reaction. After the small piece of sodium was completely dissolved, anhydrous benzene (15 mL) was added to obtain an initiator (sodium methoxide). The anhydrous benzene solution was added to the 500 mL round-bottomed flask containing benzyl-Glu-NCA through a double-ended needle, followed by adding the initiator (sodium methoxide) at a monomer molar ratio of 1/100. The reaction was carried out at room temperature for 3 days. After the reaction was completed, methanol (1000 mL) was added to precipitate a fibrous PBG. In the end, the solvent was removed via filtration and the product was placed in a vacuum oven at 40° C. to remove solvent for the next step.

Mn=173 kDa; Mw=210 kDa.

Step 3-1: Synthesis of Polypeptide Copolymer PBGA₂₀

PBG (1 g) was provided and dissolved in dichloroacetic acid (25 ml). After the PGB was completely dissolved, 33 wt % hydrobromic acid solution (0.76 mL in acetic acid) was added, and the reaction was carried out at room temperature for about 15 minutes. After the reaction was complete, diethyl ether was added to precipitate the product, and then the product was placed in a vacuum oven to remove the solvent. The product was analyzed by nuclear magnetic resonance spectroscopy. A polypeptide copolymer having a side chain hydrolysis rate of about 20% was obtained, and named PBGA₂₀.

Step 3-2: Synthesis Polypeptide Copolymer PBGA₃₀

PBG (1 g) was provided and dissolved in dichloroacetic acid (25 mL). After the PGB was completely dissolved, 33 wt % hydrobromic acid solution (0.76 mL in acetic acid) was added, and the reaction was carried out at room temperature for about 35 minutes. After the reaction was complete, diethyl ether was added to precipitate the product, and then the product was placed in a vacuum oven to remove the solvent. The product was analyzed by nuclear magnetic resonance spectroscopy. A polypeptide copolymer having a side chain hydrolysis rate of about 30% was obtained, and named PBGA₃₀.

Porous Fibrous Scaffold Fabrication

Polymer Solution Preparation

The synthesized PBG or PBGA was placed in a vacuum oven to remove the solvent and water. Then, tetrahydrofuran (THF) and dimethyl acetamide (DMAc) was provided at a weight ratio of 9:1 to form a solvent, and the PBG or PBGA was added to the solvent to form a 20 wt % polymer solution. The mixed solution was stirred and evenly dissolved for the subsequent steps.

Electrospinning

The electro-spinning apparatus used in the present invention was shown in FIG. 1, wherein the syringe was configured of a Terumo syringe (3 mL) and a 24 Gauge needle. First, the sharp tip of the needle was removed to form a flat end needle. Then, the prepared polymer solution was loaded into the syringe, and the air in the syringe was expelled. Thereafter, the syringe was fixed onto a syringe pump, and the needle was clamped by an alligator clip connecting to a high voltage power supply. Lastly, a collector was wrapped with aluminum foil and connected to the grounding alligator clip. Herein, the voltage was 20 kV, and the flow rate was 5 ml/hr. The porous fibrous scaffold made from isotropic fibers was collected using a round plate collector at a constant rotation speed of 280 rpm. The porous fibrous scaffold made from aligned fibers was collected using a roller collector at a rotation speed of 3200 rpm. After collecting the porous fibrous scaffold, the porous fibrous scaffold together with the foil were placed in a vacuum oven at 40° C. to remove the solvent.

Porous Fibrous Scaffold Properties

Fiber Width

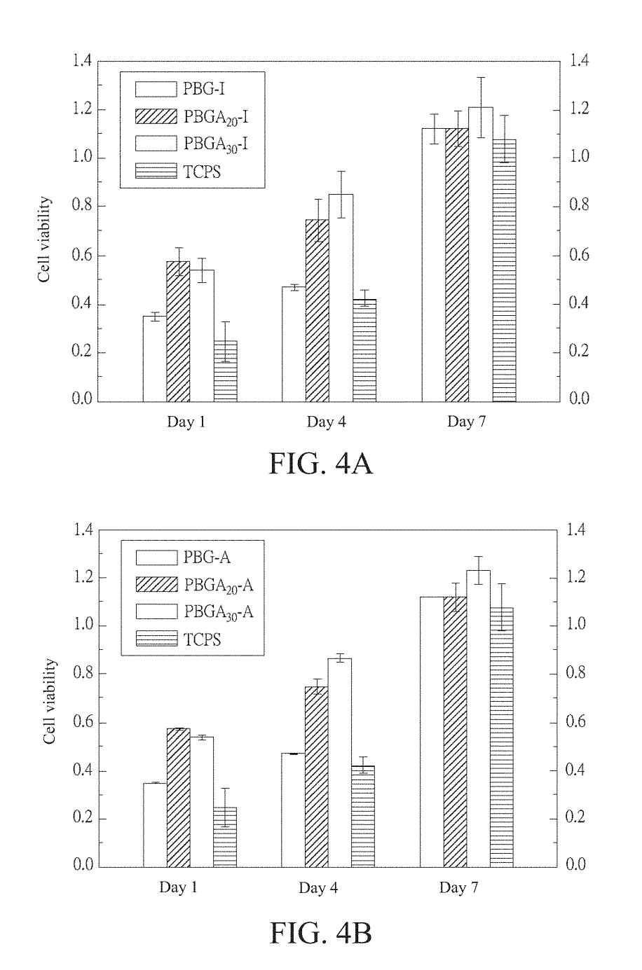

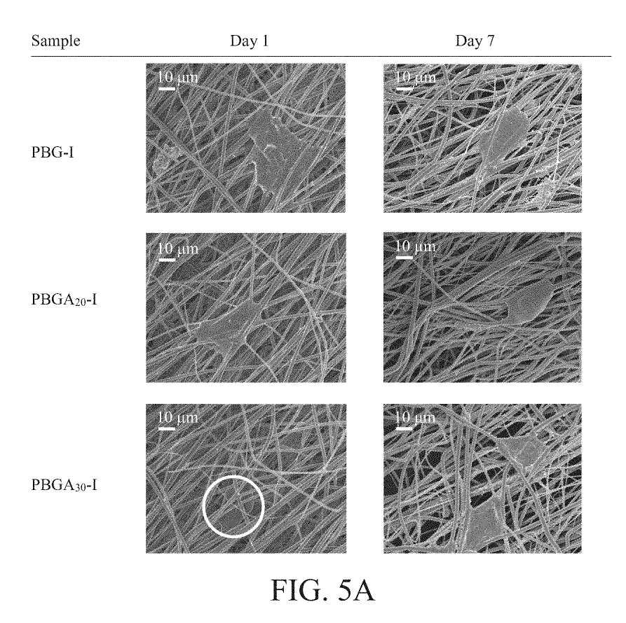

FIG. 2 is SEM images of porous fibrous scaffolds made from isotropic PBG fibers according to an embodiment of the present invention. FIG. 2(a) is an SEM image of a porous fibrous scaffold made from isotropic PBG fibers (PBG-I); FIG. 2(b) is an SEM image of a porous fibrous scaffold made from isotropic PBGA₂₀ fibers (PBGA₂₀-I); and FIG. 2(c) is an SEM image of a porous fibrous scaffold made from isotropic PBGA₃₀ fibers (PBGA₃₀-I). FIG. 3 is SEM images of porous fibrous scaffolds made from aligned PBG fibers according to an embodiment of the present invention. FIG. 3(a) is an SEM image of a porous fibrous scaffold made from aligned PBG fibers (PBG-A); FIG. 3(b) is an SEM image of a porous fibrous scaffold made from aligned PBGA₂₀ fibers (PBGA₂₀-A); and FIG. 3(c) is an SEM image of a porous fibrous scaffold made from aligned PBGA₃₀ fibers (PBGA30-A). It can be found from FIG. 2 to FIG. 3 that the fiber widths of the obtained porous fibrous scaffolds are all about 1.5 μm to 1.8 μm, and there is no significant difference in PBGA and PBG with different degrees of hydrolysis and different directionality, proving that the molecular weight of PBG does not significantly decrease with hydrolysis.

Hydrophilicity and Hydrophobicity

The present invention quantified the hydrophilicity and hydrophobicity of different scaffolds by measuring the contact angle, and the results were shown in the following Table 2. The contact angle decreases with increasing amount of glutamic acid in the polypeptide due to the acid functionality in the polymer. The contact angle also decreases when the directionality of the fiber changes from random to aligned, and thus the hydrophilicity f the porous fibrous scaffold increases.

TABLE 2

| Sample | Contact angle (°) |
|---|---|
| PBG-I | 136.1 ± 3.3 |
| PBGA₂₀-I | 127.1 ± 3.9 |
| PBGA₃₀-I | 121.8 ± 7.1 |
| PBG-A | 128.7 ± 2.8 |
| PBGA₂₀-A | 120.6 ± 2.6 |
| PBGA₃₀-A | 114.7 ± 2.4 |

Biocompatibility

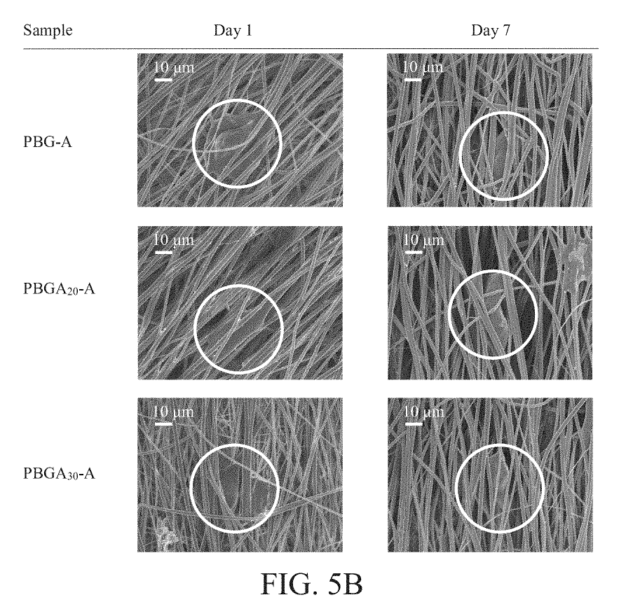

FIG. 4A is a bar chart of a biocompatibility test for porous fibrous scaffolds made from isotropic fibers according to one embodiment of the present invention; FIG. 4B is a bar chart of a biocompatibility test for porous fibrous scaffolds made from aligned fibers according to an embodiment of the present invention. Herein, tissue culture polystyrene (TCPS) serves as control group, representing that the cells is directly cultured in a plate coated with poly-L-lysine. It can be found in the FIG. 4A and FIG. 4B that the cells grew with time on the porous fibrous scaffolds either made from isotropic fibers or aligned fibers, indicating that the porous fibrous scaffolds of the present invention have excellent biocompatibility.

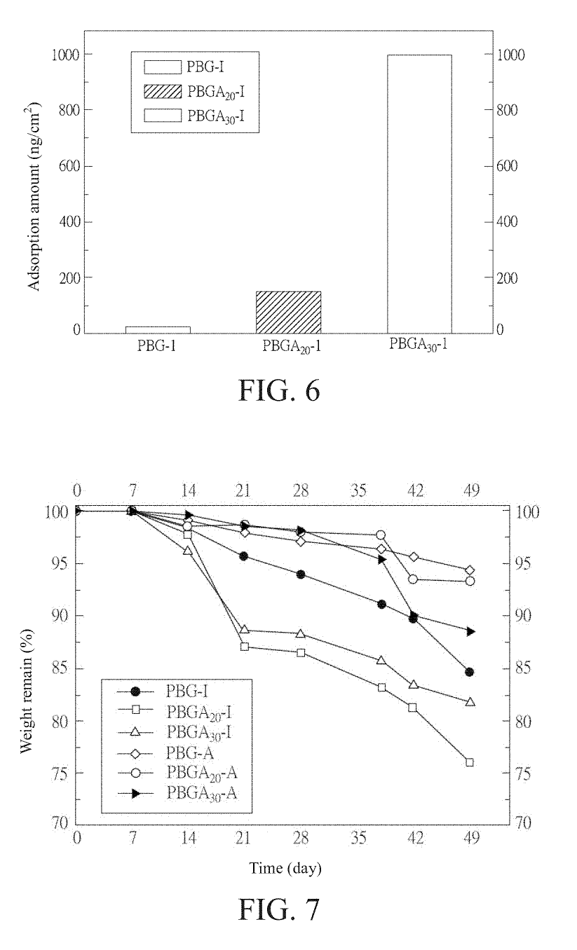

FIG. 5A is SEM images of porous fibrous scaffolds made from isotropic fibers with cells adhered thereon according to an embodiment of the present invention; and FIG. 5B is SEM images of a porous fibrous scaffolds made from aligned fibers with cells adhered thereon according to an embodiment of the present invention. As shown in FIG. 5A and FIG. 5B, since the porous fibrous scaffold has a 3D space for cell adhesion, the groups cultured with the scaffold had a higher absorbance after 7 days of culture. Furthermore, apoptosis were not observed.

Adsorption of Poly-L-Lysine by the Scaffold

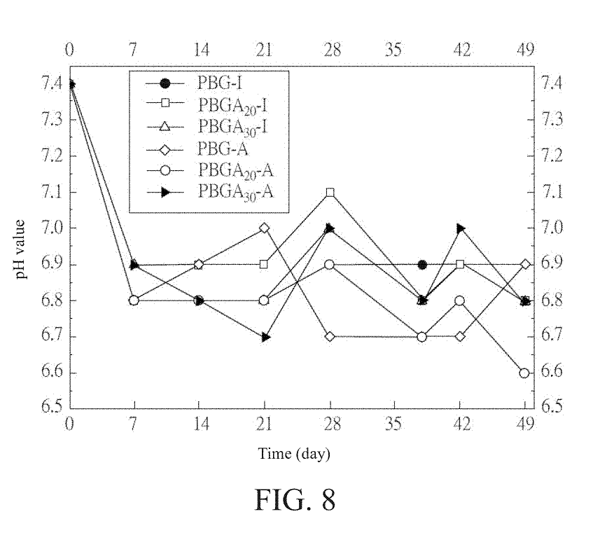

According FIG. 4A, it was found that PBGA₃₀-I had the highest cell viability in the porous fibrous scaffold made from isotropic fibers, and it was inferred that PBGA₃₀-I could adsorb more poly-L-lysine, resulting in higher cell adhesion. Therefore, the adsorption of poly-L-lysine by different porous fibrous scaffolds was measured by quartz crystal microbalance, and the results were shown in FIG. 6. The adsorption amount of PBG-I was about 20 ng/cm²; the adsorption amount of PBGA₂₀-I was about 150 ng/cm²; and the adsorption amount of PBGA₃₀-I was about 1000 ng/cm².

Biodegradability Test

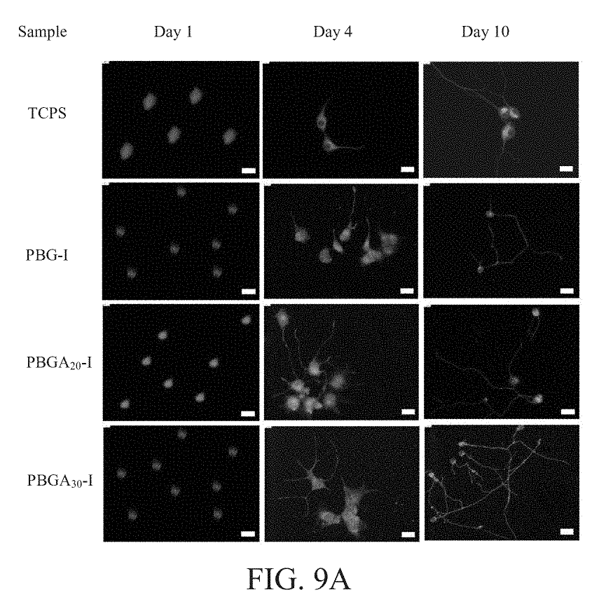

FIG. 7 is a graph showing the weight loss with time for the porous fibrous scaffolds in the biodegradability test according to an embodiment of the present invention. In the first 7 days of the test, all the porous fibrous scaffolds were not degraded, and the reason was inferred that it took time for water to diffuse into the fibers. Because of the higher level of molecular crystallization for aligned fibers, the porous fibrous scaffold made from aligned fibers was not easily degraded. Therefore, it was observed that the degradation rates of all porous :fibrous scaffolds made from isotropic fibers were faster than that of porous fibrous scaffolds made from aligned fibers. In addition, since PBGA₂₀ and PBGA₃₀ had higher hydrophilicity, it was observed that porous fibrous scaffolds made from PBGA₂₀ and PBGA₃₀ fibers had faster degradation rate than that of PBG.

Determination of pH Value After Degradation

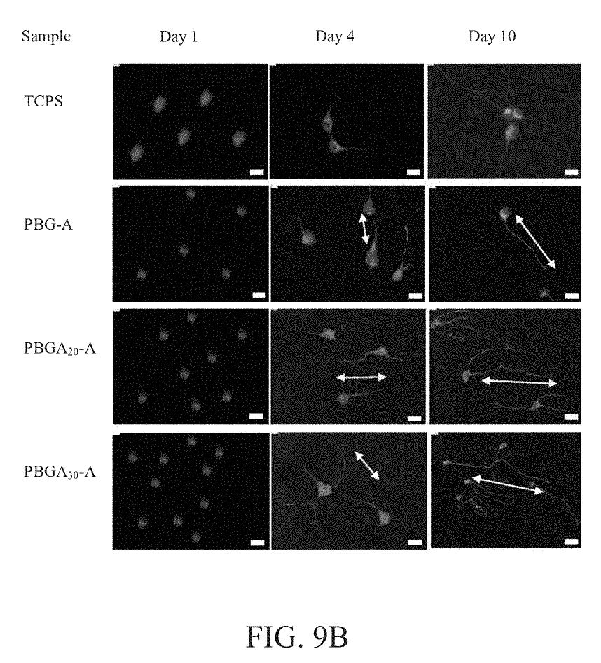

FIG. 8 is a graph showing changes in pH of a porous fibrous scaffold during degradation according to an embodiment of the present invention. On day 0, the pH was 7.4, which is the initial pH of the phosphate buffer solution. Since 5% of carbon dioxide was gradually dissolved in the water to form carbonic acid during the test, all the pH values of the solution of groups dropped to 6.8-6.9 after 7 days. After 14 days, all the pH values of the solution maintained were between 6.8 and 6.9; and after 28 days, the pH values of the solution of groups remained neutral. Therefore, it can be inferred that there was no acidic substance produced during the degradation of the porous fibrous scaffolds made from PBG and PBGA fibers, and thus the possibility of immune response might be reduced after the transplant.

Effect on Neural Differentiation

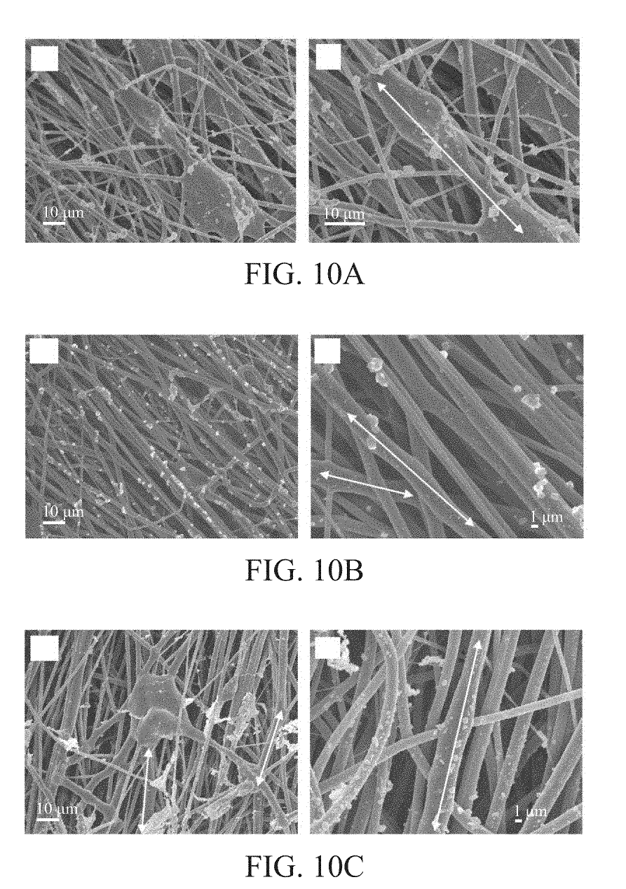

Since PC-12 cells grow neurites under the induction of nerve growth factor (NGF), the effects of different porous fibrous scaffolds on neural differentiation can be analyzed by observing the immunostaining map of the neuronal differentiation protein. FIG. 9A is images of immunofluorescence staining of PC-12 with the porous fibrous scaffolds made from isotropic fibers according to an embodiment of the present invention; FIG. 9B is images of immunofluorescence staining of PC-12 with the porous fibrous scaffolds made from aligned fibers according to an embodiment of the present invention. As shown in FIG. 9A and FIG. 9B, the cells were in the polygonal shapes on the first day of the addition of NGF, indicating that the cells were already adhesive to the substrate and the cells would differentiate to form neurites. Furthermore, in the group of porous fibrous scaffold made from aligned fibers, the cells were in a long and narrow shape. On the fourth day, the groups having porous fibrous scaffold differentiated to form more neurites; and in the groups of PBGA₂₀ and PBGA₃₀ of the present invention, the numbers of differentiated neurites were significantly larger than that of PBG group. On the 10^{th} day, it could be observed that most cells of PBGA₃₀-I group were connected with each other by neurites. Moreover, by comparing FIG. 9A with FIG. 9B, it could be seen that the neurites grew along the fiber direction in the groups having porous fibrous scaffolds made from aligned fibers since day 4.

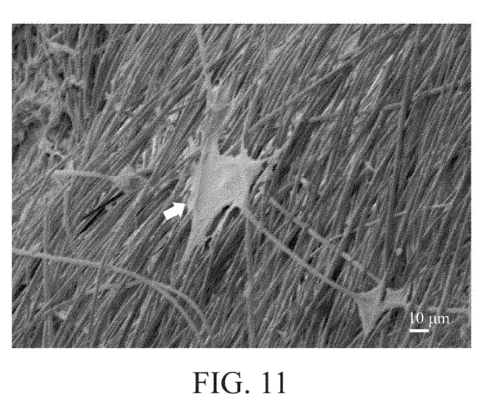

Taking PBGA₃₀-A for example, FIG. 10A to FIG. 10C are SEM images showing the cells with porous fibrous scaffolds made from PBGA₃₀-A fibers at various differentiation time point according to an embodiment of the present invention. FIG. 10A is day 1 of differentiation; FIG. 10B is day 4 of differentiation; FIG. 10C is day 10 of differentiation. Looking at FIG. 10A, neurites at short lengths were found on the first day. By enlarging the image, it could be found that all the neurites were adhered on the fibers and grew in the fiber direction. By the day 10, the phenomenon was more obvious. Accordingly, changing the fiber direction did affect the directionality of the neurite and thus lead the neurite to grow in a specific direction.

The following Table 3 shows the neurite lengths of different scaffolds, wherein the neurites lengths obtained from the PBGA₂₀ and PBGA₃₀ groups are longer. Therefore, it could be confirmed that the porous fibrous scaffolds introduced with glutamate units could stimulate neurite differentiation. Moreover, in the groups of porous fibrous scaffolds made from aligned fibers, the cells were in a long and narrow shape at the beginning of adhesion, so it facilitated neurite growth; also, the directionality of the fibers limited the number of neurite, and thus the longer neurite could be grown.

TABLE 3

| | Neurite length (μm) | | |
|---|---|---|---|
| Sample | Day 1 | Day 4 | Day 10 |
| TCPS | 5.1 ± 1.2 | 33.2 ± 15.1 | 73.1 ± 33 |
| PBG-I | 5 ± 1.9 | 32.1 ± 22.6 | 81.4 ± 33.7 |
| PBGA₂₀-I | 5.2 ± 2.2 | 39.9 ± 29.9 | 96 ± 46.3 |
| PBGA₃₀-I | 5.5 ± 1.2 | 37.6 ± 22.9 | 90.4 ± 45.8 |
| PBG-A | 5.1 ± 2.1 | 34.1 ± 23.6 | 84.4 ± 32.4 |
| PBGA₂₀-A | 5.3 ± 1.8 | 42.4 ± 23.5 | 98 ± 42.6 |
| PBGA₃₀-A | 7.8 ± 1.9 | 39.6 ± 27.3 | 94.6 ± 43.9 |

Effect on Optic Neural Differentiation

Human episomal induced pluripotent stem cell (hiPSC) derived retinal ganglion cell (RGC) progenitors was used as a model of optic nerve cells. After 7 days of differentiation, the three longest neurite lengths were taken. The average of the neurite lengths is used as a basis for comparison.

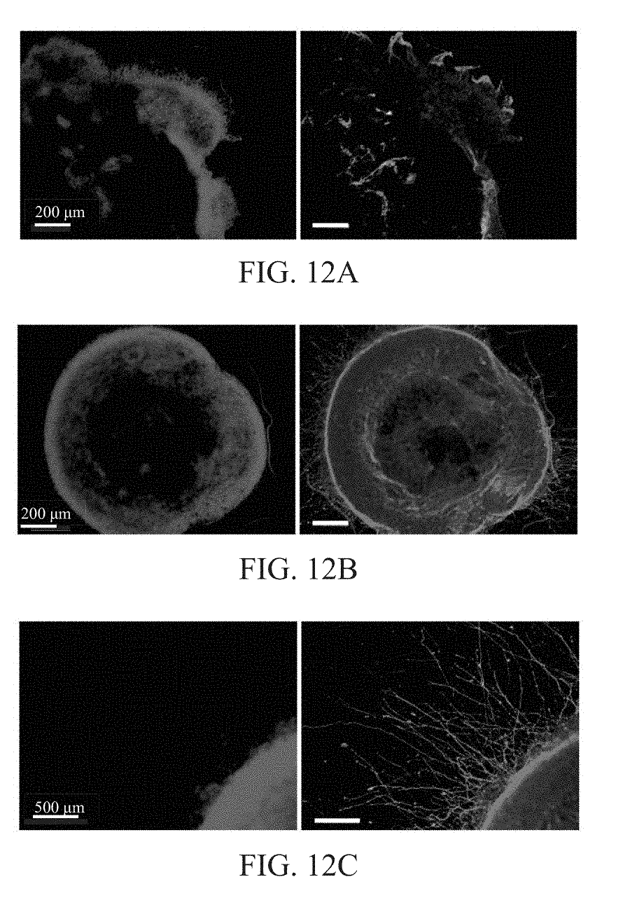

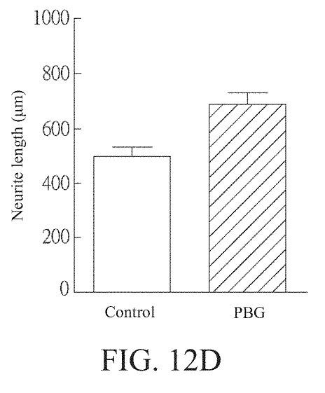

FIG. 11 is an SEM image of a hiPSC derived RGC on a porous fibrous scaffold made from PBG fibers. It could be observed from FIG. 11 that the neurites grew in the direction of the fibers. FIG. 12A to 12C are staining diagrams of a hiPSC-derived RGC optic vesicle (OV) structure according to an embodiment of the present invention, wherein the left panel was stained with DAPI and the right panel was stained with anti-βIII tubulin. Furthermore, FIG. 12A shows a control group that there is no scaffold used in this group. FIG. 12B is an experimental group cultured with a porous fibrous scaffold made from PBG fibers; and FIG. 12C is a partial enlarged view of FIG. 12B. Since βIII tubulin is expressed in the developing nerve fiber layer, the growth of neurites can be analyzed by observing the fluorescent staining of βIII tubulin. As shown in FIG. 12A to FIG. 12C, neurite growth was significantly induced in the presence of a porous fibrous scaffold made from PBG fibers, and the neurites were prominently grown by the edge of the optic vesicle structure. In addition, the neurite lengths of the control group and the group with the porous fibrous scaffold made from PBG fibers were recorded in FIG. 12D, respectively. It was confirmed that the porous fibrous scaffold made from PBG fibers did have an effect of inducing neurite growth, and therefore, it can be applied to optic nerve regeneration and/or growth.

As a result, the present invention provides a novel polypeptide copolymer, a preparation method thereof, a porous fibrous scaffold including the same. The porous fibrous scaffold can be used as a biological scaffold, has excellent biocompatibility, and stimulates neurite growth. Therefore, it can be applied to nerve regeneration and/or growth.

Although the present invention has been explained in relation to its preferred embodiment, it is to be understood that many other possible modifications and variations can be made without departing from the spirit and scope of the invention as hereinafter claimed.

What is claimed is:

1. A polypeptide copolymer, comprising: a glutamate unit; and a glutamic acid unit; wherein a ratio of a content of the glutamic acid unit to a content of the glutamate unit is in a range from 10:90 to 90:10.

2. The polypeptide copolymer according to claim 1, wherein the ratio of the content of the glutamic acid unit to the content of the glutamate unit is in a range from 10:90 to 40:60.

3. The polypeptide copolymer according to claim 1, wherein the ratio of the content of the glutamic acid unit to the content of the glutamate unit is in a range from 15:85 to 40:60.

4. The polypeptide copolymer according to claim 1, wherein the polypeptide copolymer is a random copolymer.

5. The polypeptide copolymer according to claim 1, wherein the weight average molecular weight of the polypeptide copolymer is in a range from 100 kDa to 500 kDa.

6. The polypeptide copolymer according to claim 1, wherein the glutamate unit is a benzyl glutamate unit.

7. A porous fibrous scaffold, comprising a polypeptide copolymer, which comprises: a glutamate unit; and a glutamic acid unit; wherein a ratio of a content of the glutamic acid unit to a content of the glutamate unit is in a range from 10:90 to 90:10.

8. The porous fibrous scaffold according to claim 13, wherein the porous fibrous scaffold is obtained from the polypeptide copolymer by electrospinning.

9. The porous fibrous scaffold according to claim 13, wherein the porous fibrous scaffold has a structure of unidirectional arrangement.

10. The porous fibrous scaffold according to claim 13, wherein the ratio of the content of the glutamic acid unit to the content of the glutamate unit is in a range from 10:90 to 40:60.

11. The porous fibrous scaffold according to claim 13, wherein the ratio of the content of the glutamic acid unit to the content of the glutamate unit is in a range from 15:85 to 40:60.

12. The porous fibrous scaffold according to claim 13, wherein the polypeptide copolymer is a random copolymer.

13. The porous fibrous scaffold according to claim 13, wherein the weight average molecular weight of the polypeptide copolymer is in a range from 100 kDa to 500 kDa.

14. The porous fibrous scaffold according to claim 13, wherein the glutamate unit is a benzyl glutamate unit.

15. A method for nerve regeneration or growth, comprising seeding a nerve cell onto a fibrous porous scaffold, wherein the fibrous porous scaffold comprises a glutamate unit and optionally further comprise a glutarnic acid; when the fibrous porous scaffold comprises the glutamate unit and the glutamic acid, the content of the glutamic acid unit to a content of the glutamate unit is 10:90 to 90:10.

16. The method according to claim 15, wherein the ratio of the content of the glutamic acid unit to the content of the glutamate unit is in a range from 10:90 to 40:60.

17. The method according to claim 15, wherein the ratio of the content of the glutamic acid unit to the content of the glutamate unit is in a range from 15:85 to 40:60.

18. The method according to claim 15, wherein the polypeptide copolymer is a random copolymer.

19. The method according to claim 15, wherein the weight average molecular weight of the polypeptide copolymer is in a range from 100 kDa to 500 kDa.

20. The method according to claim 15, wherein the glutamatenit is a benzyl glutamate unit.
